(19) United States
(12) Patent Application Publication
(10) Pub. No.: US 2021/0341480 A1
(43) Pub. Date: Nov. 4, 2021

(54) VIRAL TESTING IN SALIVA

(71) Applicants: Shi-Long Lu, Englewood, CO (US); Brian L. Harry, Denver, CO (US); Jose P. Zevallos, St. Louis, MO (US); Robert E. Blomquist, Boulder, CO (US); Xin Yao, Boulder, CO (US); Yue Qiu, Englewood, CO (US); Marsha T. Tharakan, Denver, CO (US)

(72) Inventors: Shi-Long Lu, Englewood, CO (US); Brian L. Harry, Denver, CO (US); Jose P. Zevallos, St. Louis, MO (US); Robert E. Blomquist, Boulder, CO (US); Xin Yao, Boulder, CO (US); Yue Qiu, Englewood, CO (US); Marsha T. Tharakan, Denver, CO (US)

(21) Appl. No.: 17/244,333

(22) Filed: Apr. 29, 2021

Related U.S. Application Data
(60) Provisional application No. 63/017,354, filed on Apr. 29, 2020.

(51) Int. Cl. G01N 33/569; G01N 33/68; C12Q 1/70; C12Q 1/686

(52) U.S. Cl. CPC G01N 33/56983; G01N 33/6854; G01N 2333/165; C12Q 1/686; C12Q 1/70

(57) ABSTRACT

Saliva-based testing for viruses including SARS-CoV-2 are provided. Simple collection methods allow for at-home collection, reducing the risk and burden on healthcare workers using conventional testing methods. Tests can quantitatively analyze both viral nucleic acids to assess viral load as well as virus-specific antibodies to track disease progression and potential immunity.

### RELATED APPLICATIONS

This application claims the benefit of and priority to U.S. Provisional Application No. 63/017,354, filed Apr. 29, 2020, the content of which is incorporated by reference herein.

### FIELD OF THE INVENTION

The invention provides compositions and methods for detecting viruses in body fluids.

### BACKGROUND

Early and reliable viral surveillance is critical in controlling community spread of viral pathogens. For example, the lack of reliable testing hampered the response to the 2019 novel coronavirus pandemic. The majority of COVID-19 tests in the United States with FDA Emergency Use Authorization (EUA) utilize nasopharyngeal swabs. Nasopharyngeal collection is invasive, technically challenging, and has reported high false-negative rates, approaching 30-40%.

Most viral testing requires significant technical know-how and must be administered by healthcare professionals. Anyone suspected of being infected must leave their homes and come in contact with at least a healthcare worker administering the test. Accordingly, conventional testing risks viral spread and commands scarce resources, including personal protective equipment.

In light of the resource constraints attendant to widespread viral testing, there is a need in the art for rapid, convenient, safe, and reliable testing in order to limit spread of pervasive viral pathogens.

### SUMMARY OF THE INVENTION

Compositions and methods of the invention provide saliva-based testing for viral pathogens. Tests of the invention allow for the detection of viral infection and the presence of viral antibodies in saliva, thereby enabling individuals to self-test without the assistance of a healthcare worker. The invention results in efficiencies with respect to personnel, equipment and time, while providing highly sensitive and specific viral surveillance.

The present invention is especially useful to detect respiratory viral infections. As a reservoir for the upper aerodigestive tract, saliva provides a good representative sampling for the assessment of both the presence of a virus and viral load. For example with respect to the SARS-CoV-2 virus, recent studies have found viral concentration in saliva to be among the highest in tested body fluids. See, To K K, Tsang O T, Chik-Yan Yip C, et al., 2020, Consistent detection of 2019 novel coronavirus in saliva, Clinical infectious diseases: an official publication of the Infectious Diseases Society of America; Wang W, Xu Y, Gao R, et al., 2020, Detection of SARS-CoV-2 in Different Types of Clinical Specimens, JAMA; the content of each of which is incorporated herein by reference.

Saliva can be self-collected at home by patients, avoiding the need for direct contact with medical workers and the associated risk of disease transmission. Compositions and methods of the invention are useful to detect viral nucleic acid in saliva. In a preferred embodiment, methods of the invention comprise the use of PCR to amplify and detect viral nucleic acids in saliva.

In preferred embodiments, digital PCR and specifically droplet digital PCR (ddPCR), is performed on a saliva sample to detect viral nucleic acid (RNA or DNA, depending on the type of virus). Quantitative methods such as digital PCR or quantitative fluorescence-labelled PCR (qPCR) can be used to not only diagnose infection but quantify viral load and provide valuable information on disease prognosis and progression for the tested patient. Digital PCR can provide a higher sensitivity than qPCR and is therefore preferred. Recent studies of ddPCR testing for SARS-CoV-2 in NPS samples have shown promise, delivering high sensitivity and accuracy with reduced false negative reports compared to real-time PCR (RT-PCR) analysis. See Tao S, et al., 2020, ddPCR: a more sensitive and accurate tool for SARS-CoV-2 detection in low viral load specimens, medRxiv (preprint). https://doi.org/10.1101/2020.02.29.20029439; Dong, I et al., 2020, Highly accurate and sensitive diagnostic detection of SARS-CoV-2 by digital PCR. medRxiv (preprint). https://doi.org/10.1101/2020.03.14.20036129; the content of reach of which is incorporated herein by reference.

In certain embodiments, screening accuracy may be supplemented by the addition of antibody testing in saliva. The ability to detect viruses using two different methods (nucleic acid and antibody detection) in a single saliva sample provides a robust and accurate test that can be self-administered. Specifically, compositions and methods of the invention can be used to detect virus-specific IgA and IgG antibodies (known to be present in mucosal secretions) and provide a valuable metric of both mucosal (IgA) and systemic (IgG) immunity. The present combination of PCR-based viral detection and “saliva serology” (antibody detection) maximizes diagnostic information from a single saliva sample and provides a useful tool in detecting both current infection and past exposure and immunity. Accordingly, tests of the invention can play a valuable role in the easing of social distancing requirements by providing detailed information on disease exposure, infection, and potential immunity with minimal risk of additional transmission.

In certain embodiments, quantification of viral load and/or antibodies are monitored longitudinally in patient samples to provide information regarding disease progression and to predict outcomes such as likelihood of intubation, ICU admission, discharge, and death as well as time until intubation, ICU admission, or discharge. In cases of bed, material, or equipment shortages, the ability to predict the likelihood and timing of the above outcomes can help ration and plan for patient housing and treatment.

Methods of the invention take advantage of the fact that respiratory viruses are present in saliva and salivary tissue. Accordingly, the invention is applicable to the detection of any respiratory virus, including but not limited to, members of the paramyxoviridae, picornaviradae, coronaviridae, parvoviridae, and enteroviridae families.

### BRIEF DESCRIPTION OF THE DRAWINGS

FIG. 1A shows qualitative detection by agarose gel electrophoresis.

FIG. 1B shows quantitative results obtained by qRT-PCR.

FIG. 2 Shows DNA stability in saliva stored at various temperatures over times of 1, 3, and 7 days.

FIG. 3 shows Ct counts for qPCR with primer-probe sets targeting the N1 region of SARS-CoV-2 for saliva and nasal swab samples.

FIG. 4 shows Ct counts for qPCR with primer-probe sets targeting the N2 region of SARS-CoV-2 for saliva and nasal swab samples.

FIG. 5 shows Ct counts for an internal control host gene ribonuclease P (RNP) for saliva and nasal swab samples.

FIG. 6 shows Ct counts for qPCR with primer-probe sets targeting the N1 region of SARS-CoV-2 for extraction-free saliva and extracted nasal swab samples.

FIG. 7 shows Ct counts for qPCR with primer-probe sets targeting the N2 region of SARS-CoV-2 for extraction-free saliva and extracted nasal swab samples.

FIG. 8 shows Ct counts for an internal control host gene ribonuclease P (RNP) for extraction-free saliva and extracted nasal swab samples.

FIG. 9 shows copies/μL of SARS-CoV-2 in nasal swab and saliva samples as determined for low medium, and high viral loads using N1-targeted primers.

FIG. 10 shows copies/μL of SARS-CoV-2 in nasal swab and saliva samples as determined for low medium, and high viral loads using N2-targeted primers.

FIG. 11 compares Ct values obtained from qPCR testing to copies/μL values obtained using ddPCR performed on the same nasal swab samples using N1-region-targeting primers.

FIG. 12 compares Ct values obtained from qPCR testing to copies/μL values obtained using ddPCR performed on the same nasal swab samples using N2-region-targeting primers.

FIG. 13 compares Ct values obtained from qPCR testing to copies/μL values obtained using ddPCR performed on the same nasal swab samples using RPP30-targeting primers.

FIG. 14 compares Ct values obtained from qPCR testing to copies/μL values obtained using ddPCR performed on the same saliva samples using N1-region-targeting primers.

FIG. 15 compares Ct values obtained from qPCR testing to copies/μL values obtained using ddPCR performed on the same saliva samples using N2-region-targeting primers.

FIG. 16 compares Ct values obtained from qPCR testing to copies/μL values obtained using ddPCR performed on the same saliva samples using RPP30-targeting primers.

FIG. 17 shows regression modeling comparing paired ddPCR and qPCR of nasal swab samples using N1-region-targeting primers.

FIG. 18 shows regression modeling comparing paired ddPCR and qPCR of nasal swab samples using N2-region-targeting primers.

FIG. 19 shows regression modeling comparing paired ddPCR and qPCR of nasal swab samples using RNP control primers.

FIG. 20 shows regression modeling comparing paired ddPCR and qPCR of saliva samples using N1-region-targeting primers.

FIG. 21 shows regression modeling comparing paired ddPCR and qPCR of saliva samples using N2-region-targeting primers.

FIG. 22 shows regression modeling comparing paired ddPCR and qPCR of saliva samples using RNP control primers.

FIG. 23 shows a comparison of longitudinal testing of saliva and nasal swab samples in SARS-CoV-2 patients.

### DETAILED DESCRIPTION

Compositions and methods of the invention relate to viral detection in saliva. Particularly, one or more of nucleic acid analysis (e.g., through ddPCR) and antibody testing (e.g., IgA and IgA testing) for past or present COVID-19 infections are contemplated. The ability to provide robust, quantified information through both nucleic acid profiling and “saliva serology” analysis from a self-collected saliva sample provides more accurate and therefore actionable data than current tests while avoiding the transmission risks associated with tests that require medical worker administration.

Tests of the invention may be used to detect any viral infection. Primers and methods (e.g., reverse transcription and amplification for RNA detection) can be selected to target specific sequences of DNA or RNA viruses for detection in saliva using PCR methods. Tests may target, for example, RSV, influenza, parainfluenza virus, HPV, HIV, Hepatitis, cytomegalovirus, Epstein-Barr virus, rhinoviruses, and adenovirus. In various embodiments, a plurality of different antibody assays and primer sets may be used in a multiplex analysis on a single saliva sample to detect the presence of multiple viruses. In certain embodiments, tests of the invention may be used to detect viruses associated with respiratory infections or mucosal infections where saliva may be a significant reservoir of viral material. Tests of the invention may be targeted to detect coronavirus nucleic acids and coronavirus-specific antibodies. Exemplary coronavirus primer targets for PCR-based detection include sequences in the N, ORF1ab, and E genes. In preferred embodiments, tests of the invention target SARS-CoV-2 and may be used to detect and monitor current or past COVID-19 infection and treatment thereof.

A significant advantage of the current invention is the ability to provide quantitative analysis of viral load and antibody production that can be used to stage and track patient progress, predict patient outcomes, and gauge response to various therapeutic treatments. Viral load and antibody levels may be tracked in samples collected over time to track disease progress and/or may be compared to standard threshold levels to aid in predicting outcomes. Threshold levels may be determined from a pool of prior patients and may be tailored to the patient based on common demographics, medical history, and other metrics. Levels may be normalized in various embodiments. For example, IgA and/or IgG antibody levels for virus-specific antibodies may be normalized against all IgA and/or IgG antibodies detected in the saliva sample.

Saliva samples may be collected from patients by, for example, having them spit into a provided sterile container. With simple instructions, a patient can provide the required saliva sample while in isolation without the need to visit a medical worker. A box or envelope may be provided with the sterile container and instructions such that a patient can, after proving the sample and sealing the container, transfer the container to a laboratory for testing. The nucleic acid and/or antibody assays discussed below can then be performed in a controlled laboratory setting with minimal risk of exposure. Scarce PPE equipment and medical personnel resources can accordingly be conserved.

Tests of the invention have multiple applications including testing for current infection, past exposure, disease severity and staging, and outcome prediction. Additional applications can include tests for pregnant women to assess vertical transmission risks and test for potential blood donors to assess horizontal transmission risks.

### Nucleic Acid Analysis

Tests of the invention may include PCR-based analysis of viral nucleic acids in saliva samples collected from patients. Simple PCR analysis may include amplifying DNA (that may be reverse-transcribed from viral RNA) with virus-specific antibodies and detecting bands of the predicted size using gel electrophoresis. However, in preferred embodiments, a quantitative PCR method is used to provide information not only on the presence of viral nucleic acids but the viral load as indicated by the amount of viral nucleic acid in the sample. A preferred quantitative PCR method is dPCR.

Digital polymerase chain reaction (dPCR) is a refinement of conventional polymerase chain reaction methods that can be used to directly quantify and clonally amplify nucleic acids strands including DNA, cDNA, or RNA. In dPCR a sample is separated into a large number of partitions and the reaction is carried out in each partition individually, thereby permitting sensitive quantification of target DNA through fluorescence analysis in each partition as opposed to a single value for the entire sample as found in standard PCR techniques.

Droplet Digital PCR (ddPCR) is a method of dPCR wherein the aforementioned partitions consist of nanoliter-sized water-oil emulsion droplets in which PCR reactions and fluorescence detection can be performed using, for example, droplet flow cytometry. The methods for creating and reading droplets for ddPCR have been described in detail elsewhere (see Zhong et al., ‘Multiplex digital PCR: breaking the one target per color barrier of quantitative PCR’, Lab Chip, 11:2167-2174, 2011), but in essence each droplet is like a separate reaction well and, after thermal cycling, the fluorescence intensities of each individual droplet were read out in a flow-through instrument like a flow cytometer that recorded the peak fluorescence intensities.

While compositions and methods of the invention may be used to detect nucleic acid and/or antibodies specific to any virus, in preferred embodiments, SARS-CoV-2 is the detection target. Exemplary primers and probes for the detection of SARS-CoV-2 have been disclosed by the Chinese CDC (targeting the N and ORF1ab genes) and the WHO (targeting the E gene) and are provided in Tao S, et al., 2020 and Dong, I et al. 2020. Compositions and methods of the invention for the detection of COVID-19 infection using ddPCR of saliva samples contemplate using the same primers and probes discussed therein.

ddPCR detection and quantification of SARS-CoV-2 in NPS samples has been successfully demonstrated and such testing is currently being offered by, for example, Biodesix (Boulder, Colo.). Compositions and methods of the invention apply similar ddPCR techniques but to a more easily-obtained saliva sample.

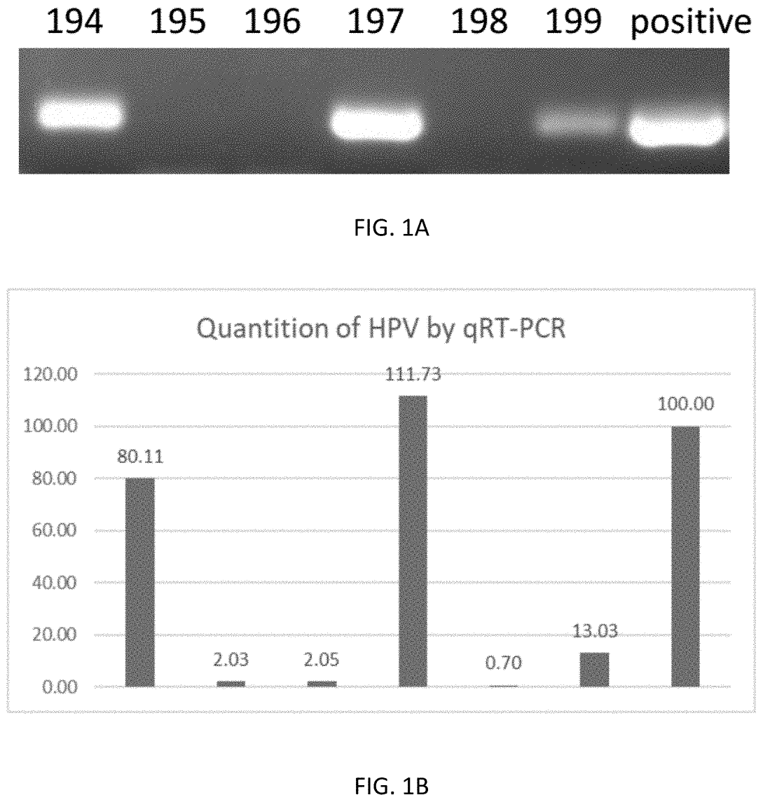

As mentioned above, saliva has been found to provide a good representation of viral load with respect to COVID-19 infections. See, To K K, et al., 2020; Wang W, et al., 2020. Recognizing this underexploited source of viral nucleic acids in infected patients, compositions and methods leverage ddPCR to detect and quantify those nucleic acids. The feasibility PCR-based DNA analysis in saliva has been demonstrated for tumor DNA, as discussed above. Various viral DNA has also be detected in saliva including, for example, human papillomavirus (HPV) DNA. FIGS. 1A and 1B demonstrate successful detection and quantification of HPV using qRT-PCR from saliva samples from head and neck cancer patients. FIG. 1A shows qualitative detection by agarose gel electrophoresis and FIG. 1B shows quantitative results obtained by qRT-PCR.

Using the quantitative methods of the invention, viral load can be longitudinally monitored for patients to assess disease progression and determine therapeutic effect of various treatments. For example, a comparison along the disease process will provide useful information for public safety and patient care. Samples may be collected at irregular or regular intervals to establish longitudinal data. For example, saliva may be collected from a patient multiple times in a day, at least daily, at least every two days, at least every three days, at least every four days, at least every five days, at least every six days, at least every week. Such data can be used to guide treatment decisions or predict clinical outcomes. Examples of predicted outcomes may include intubation, ICU admission, recovery, death, and the timing of any of the above.

Viral load can be normalized as copies of virus against copies of a host gene internal control. Patients' demographic information, including race and socioeconomic data, and clinical parameters including past medical history and medical management can be analyzed with regard to viral load to identify links between viral load and the above parameters. Such information can also be used to tailor outcome predictions based on viral load by comparing the patient to a database of patients with similar attributes. Such information may also be used to identify at-risk populations.

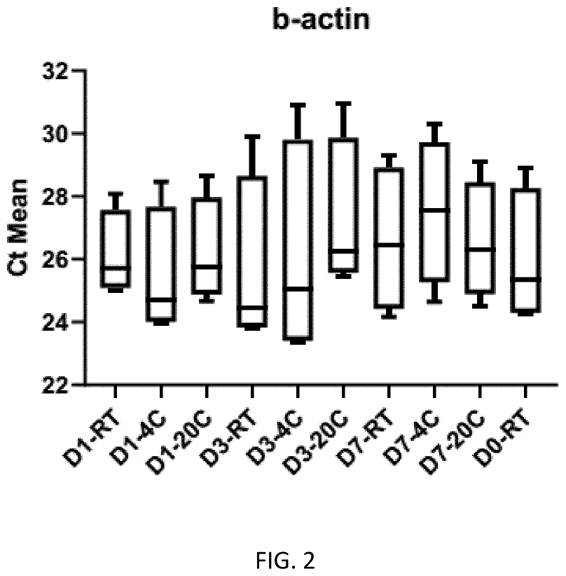

Time from collection to testing and sample storage may be important in providing accurate viral load data. DNA stability in saliva has been examined at different temperatures (RT: room temperature, 4 C, and −20 C) over periods of 1, 3, and 7 days and compared to fresh saliva at room temperature (DO-RT) with results shown in FIG. 2. Amplification efficacy of b-actin (Ct mean) was used for the evaluation. As shown, DNA is relatively stable, even at room temperature, for periods of a week or more. Accordingly, simple and inexpensive self-collection methods including at-home kits and simple packaging can be effectively used to obtain, transfer, and store samples for testing.

### Antibody Testing

While most current SARS-CoV-2 tests focus on nucleic acid, serological tests are now emerging to examine antibodies against SARS-CoV-2. Such testing is useful in understanding immunity, tracking exposure, and guiding the eventual relaxation of social distancing or return-to-work campaigns. Serological tests mostly focus on production of antiviral IgM and IgG antibodies. Tests of the invention preferably include IgA antibody analysis. The upper aerodigestive tract is lined with mucosal membranes, and is the primary site of SARS-CoV-2 infection. As the principal antibody class in mucosal secretions, IgA is the first line of defense against respiratory viruses. IgG, which appears later in the immune response, is a metric of the systemic immunity normally measured in serum. However, IgG is also found in saliva and a high correlation between blood and salivary IgG has been shown, supporting the feasibility of developing a “saliva serology” test as presently described. See Hettegger P, Huber J, Passecker K, et al., 2019, High similarity of IgG antibody profiles in blood and saliva opens opportunities for saliva based serology, PLoS One, 14(6), incorporated herein by reference. The presently described saliva antibody test can detect anti-SARS-CoV-2 IgA and IgG present in saliva and provide a more comprehensive evaluation of both mucosal and systemic immunity against COVID-19.

Compared to nucleic acids, proteins are relatively less stable and more sensitive to temperature and storage. Furthermore, salivary proteins may be more susceptible to degradation compared to serum proteins. See, Dawes C, Wong D T W. 2019, Role of Saliva and Salivary Diagnostics in the Advancement of Oral Health, J Dent Res, 98(2):133-141, incorporated herein by reference. Accordingly, in tests where antibody analysis is used, instructions and materials may be tailored to promote protein stability including insulated packaging and rapid shipment and testing methods.

In preferred embodiments, an ELISA-based assay may be used for detection and quantitation of salivary IgA and IgG specific to SARS-CoV-2. ELISA assays for detecting SARS-CoV-2-specific antibodies in blood are available, for example, from Eurolmmun AG (Lubeck, Germany). Dynamic changes in antiviral IgA and IgG in saliva can be monitored throughout progressive stages of infection and immunity as discussed with respect to nucleic-acid-derived viral load above. Accordingly, longitudinal antibody data can be similarly used for outcome prediction as well as disease progression and therapeutic effectiveness monitoring. Additionally, antibody levels may be monitored after discharge to aid in predicting immunity. Quantitation of anti-SARS-CoV-2 IgA and IgG may be performed by normalizing the amount of anti-SARS-CoV-2 antibody to total antibody in saliva or to the total IgA and IgG present (respectively). As with viral load data, antibody information may be correlated with basic demographic (e.g. age, sex, race) and clinical information (e.g. pre-existing conditions, clinical course, outcomes) and patterns identified therein may be used to tailor outcome predictions. Any biomarker-based immunoassay may be used in the invention for the detection of viral-associated antibodies (e.g., a stick-dip test, a nitrocellulose strip, or any lateral flow immunoassay). Some non-limiting preferred examples include a bead-based assay, a luminescent assay, a metal-linked immunosorbent assay, or a point-of-care immunochromatographic assay.

IgA is expected to become detectable within 10 days and IgG is expected to become detectable within 28 days. Accordingly, in certain embodiments, the relative levels of IgA and IgG antibodies may be used to determine the initial date of infection.

Antibody assays of the invention may rely on the receptor-binding domain (RBD) or S1 subunit of the spike protein, which are thought to confer additional specificity compared to other coronavirus antigens. See, Okba N M A, Muller M A, Li W, et al., 2020, Severe Acute Respiratory Syndrome Coronavirus 2-Specific Antibody Responses in Coronavirus Disease 2019 Patients, Emerging infectious diseases, 26(7), incorporated herein by reference.

### EXAMPLES

### Example 1—qPCR Comparison of SARS-CoV-2 from Saliva and Nasal Swab

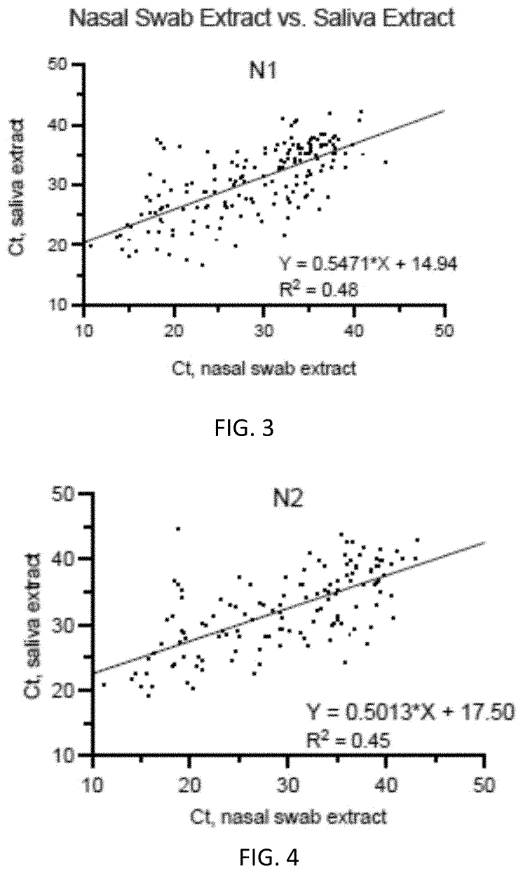

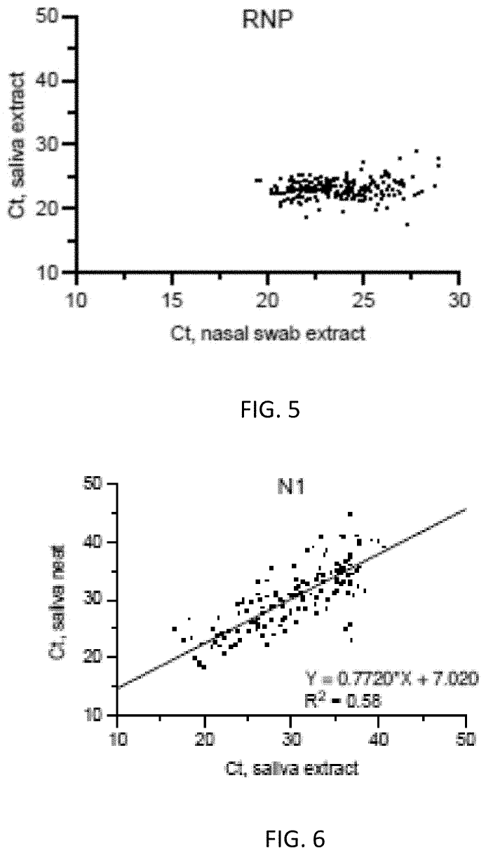

Saliva and nasal swab samples were tested via qPCR with resulting Ct or Cq values shown. FIG. 3 shows Ct counts for qPCR with primer-probe sets targeting the N1 region of SARS-CoV-2 for saliva and nasal swab samples. FIG. 4 shows Ct counts for qPCR with primer-probe sets targeting the N2 region of SARS-CoV-2 for saliva and nasal swab samples. FIG. 5 shows Ct counts for an internal control host gene ribonuclease P (RNP) for saliva and nasal swab samples. For each of FIGS. 3-5, each data point represents a single source from which both nasal swab and saliva samples were prepared. Both the saliva and nasal swab samples were subjected to RNA extraction prior to qPCR.

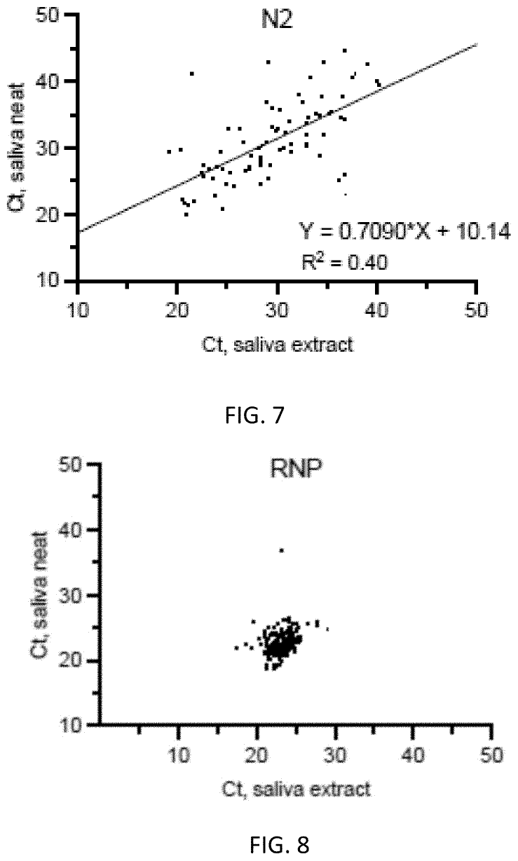

FIGS. 6-8 show the same comparisons but with the nasal swab subjected to RNA extraction and the saliva prepared for rapid PCR with no RNA extraction. Examples of such RNA-extraction free methods are described, for example, in U.S. Prov. Pat. App. 63/158,685, incorporated herein by reference. FIG. 6 shows Ct counts for qPCR with primer-probe sets targeting the N1 region of SARS-CoV-2 for extraction-free saliva and extracted nasal swab samples. FIG. 7 shows Ct counts for qPCR with primer-probe sets targeting the N2 region of SARS-CoV-2 for extraction-free saliva and extracted nasal swab samples. FIG. 8 shows Ct counts for an internal control host gene ribonuclease P (RNP) for extraction-free saliva and extracted nasal swab samples. As illustrated, the N1 target in samples containing SARS-CoV-2 was consistently measured qualitatively and quantitatively in saliva with or without extraction of nucleic acids when compared to standard nasal swab methods. Those findings are backed up by the consistent measurement of the internal control RNP in saliva with or without extraction methods.

### Example 2—ddPCR Precision Testing

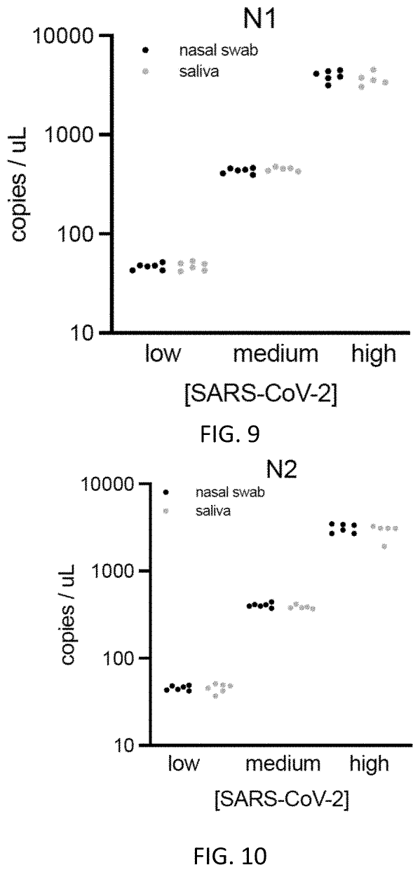

N1 and N2 targeted ddPCR using the BioRad SARS-CoV-2 ddPCR assay was performed on saliva and nasal swab samples having various viral loads. ddPCR gives an absolute viral load measurement in copies/μL. FIGS. 9 and 10 show the reproducibility and accuracy of ddPCR testing in saliva and nasal swab testing. FIG. 9 shows copies/μL of SARS-CoV-2 in nasal swab and saliva samples as determined for low medium, and high viral loads using N1-targeted primers. FIG. 10 shows copies/μL of SARS-CoV-2 in nasal swab and saliva samples as determined for low medium, and high viral loads using N2-targeted primers.

### Example 3—qPCR and ddPCR Comparison

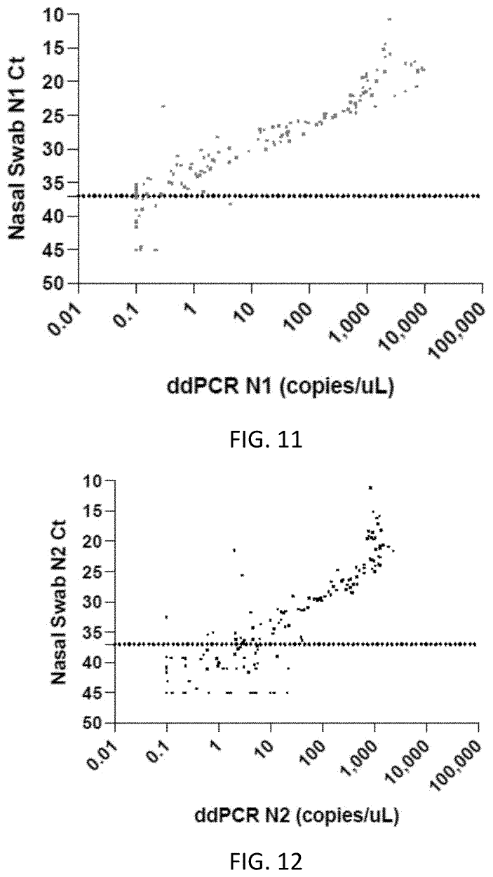

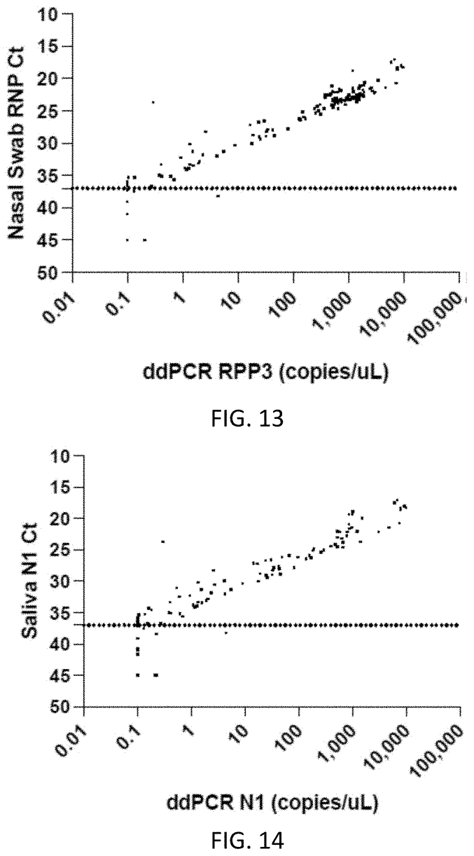

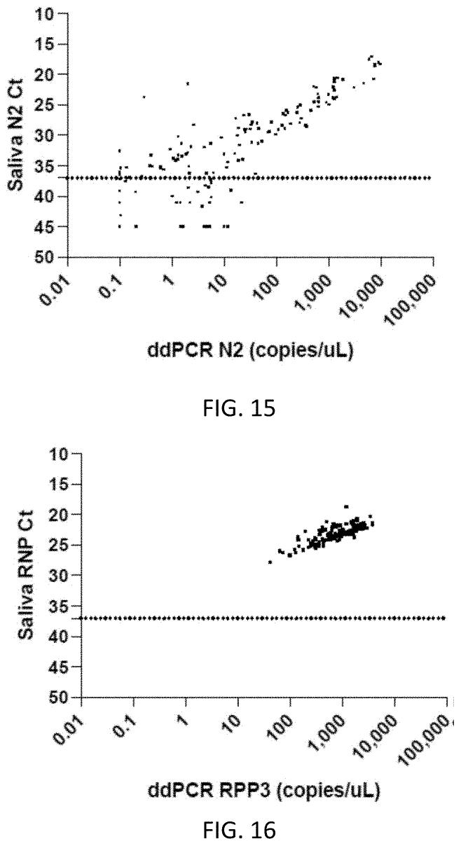

Nasal swab and saliva samples were tested using ddPCR and qPCR methods targeting SARS-CoV-2 N1 or N2 regions or the RPP30 control gene and the results were compared. FIG. 11 compares Ct values obtained from qPCR testing to copies/μL values obtained using ddPCR performed on the same nasal swab samples using N1-region-targeting primers. FIG. 12 compares Ct values obtained from qPCR testing to copies/μL values obtained using ddPCR performed on the same nasal swab samples using N2-region-targeting primers. FIG. 13 compares Ct values obtained from qPCR testing to copies/μL values obtained using ddPCR performed on the same nasal swab samples using RPP30-targeting primers. FIG. 14 compares Ct values obtained from qPCR testing to copies/μL values obtained using ddPCR performed on the same saliva samples using N1-region-targeting primers. FIG. 15 compares Ct values obtained from qPCR testing to copies/μL values obtained using ddPCR performed on the same saliva samples using N2-region-targeting primers. FIG. 16 compares Ct values obtained from qPCR testing to copies/μL values obtained using ddPCR performed on the same saliva samples using RPP30-targeting primers. In both saliva and nasal swab samples, the Ct value obtained via qPCR testing proves a good surrogate for viral load as determined by ddPCR.

Paired results for ddPCR and qPCR testing for N1, N2, and RNP for both swab and saliva samples were further analyzed to assess the associations therebetween. For most of the paired data, segment or piece-wised linear regression was used to assess the association. R package ‘segmented’ was used for the analysis, which used the Bayesian information criteria BIC to select the best fitted models.

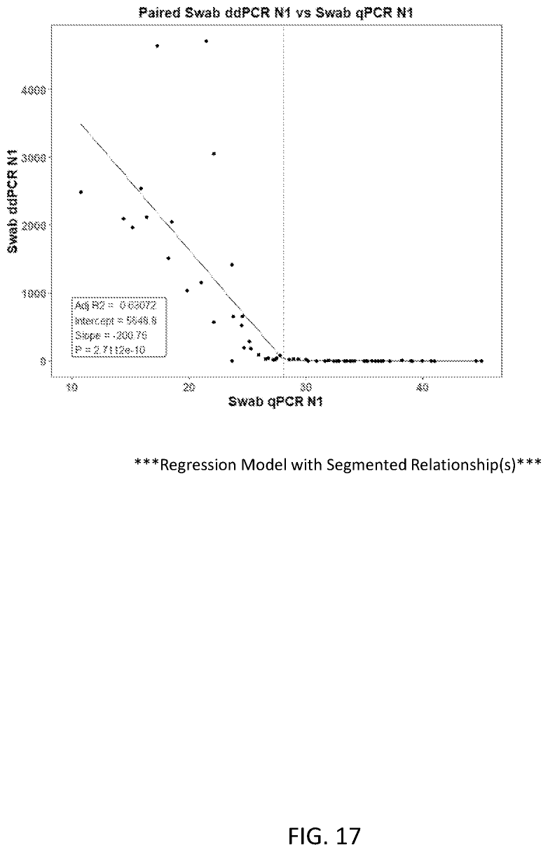

FIG. 17 shows regression modeling comparing paired ddPCR and qPCR of nasal swab samples using N1-region-targeting primers using the following model:

Y=Swab dd N1, X=swab_q_N1

Y=5648.8−200.8*X(X<=28.09)−0.737*X(X>=28.1)

Multiple R-Squared: 0.6478, Adjusted R-squared: 0.6307.

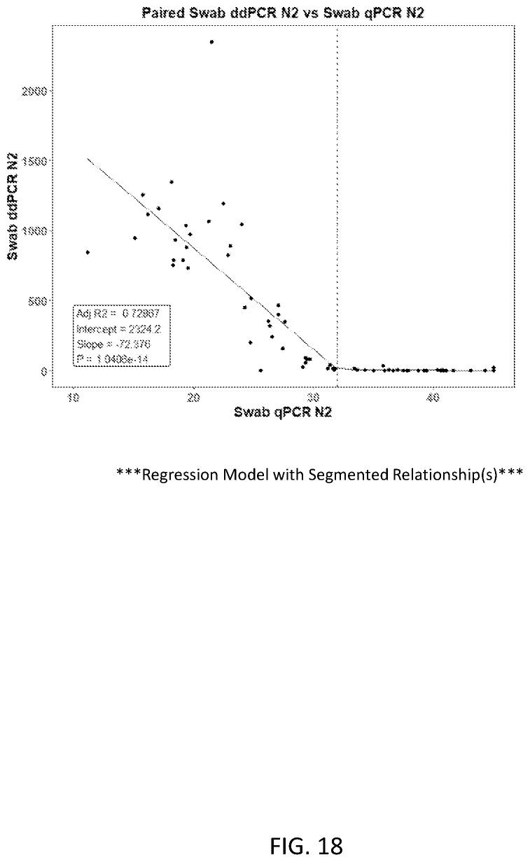

FIG. 18 shows regression modeling comparing paired ddPCR and qPCR of nasal swab samples using N2-region-targeting primers using the following model:

Y=Swab ddPCR N2, x=swab_q_N2

Y=2324.2−72.38*X(X<=32.0)−0.557*X(X>32)

Multiple R-Squared: 0.7398, Adjusted R-squared: 0.7287.

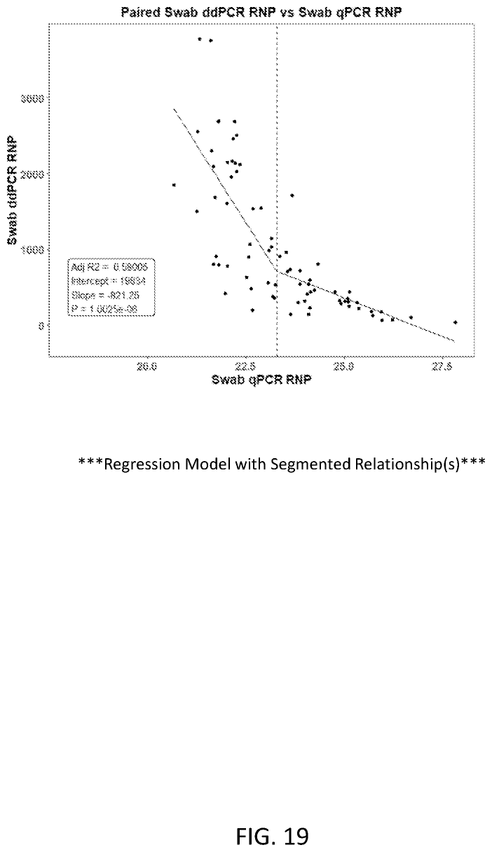

FIG. 19 shows regression modeling comparing paired ddPCR and qPCR of nasal swab samples using RNP control primers using the following model:

Y=Swab dd RNP, x=swab_q_RNP

Y=19833.9−821.2*X(X<=23.285)−204.80*X(X>23.285)

Multiple R-Squared: 0.5973, Adjusted R-squared: 0.58

Alternative model: a degree of 2 polynomial regression Multiple R-squared: 0.5878, Adjusted R-squared: 0.5762.

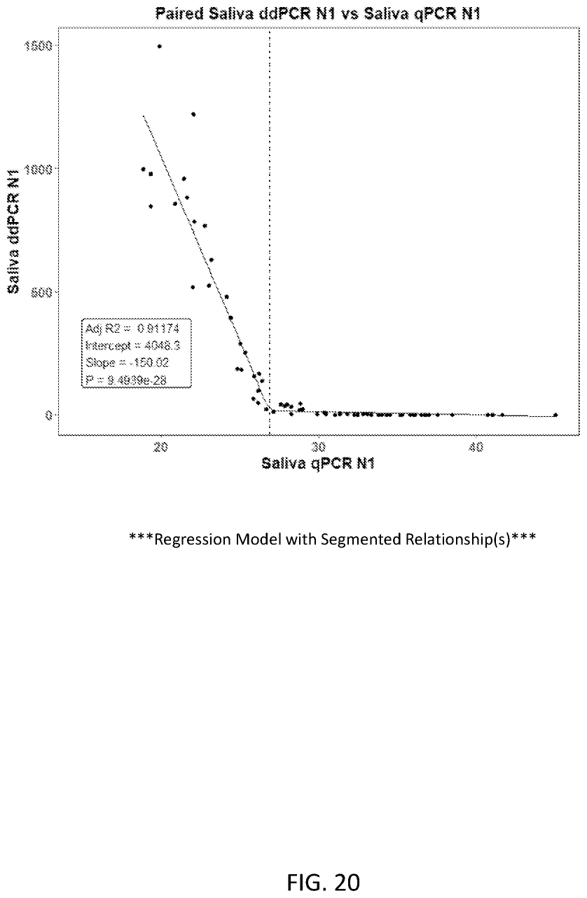

FIG. 20 shows regression modeling comparing paired ddPCR and qPCR of saliva samples using N1-region-targeting primers using the following model:

Y=saliva dd N1, x=saliva q N1

Y=4048.258−150.023*X(X<=26.87)−1.324*X(X>26.87)

Multiple R-Squared: 0.9154, Adjusted R-squared: 0.9117.

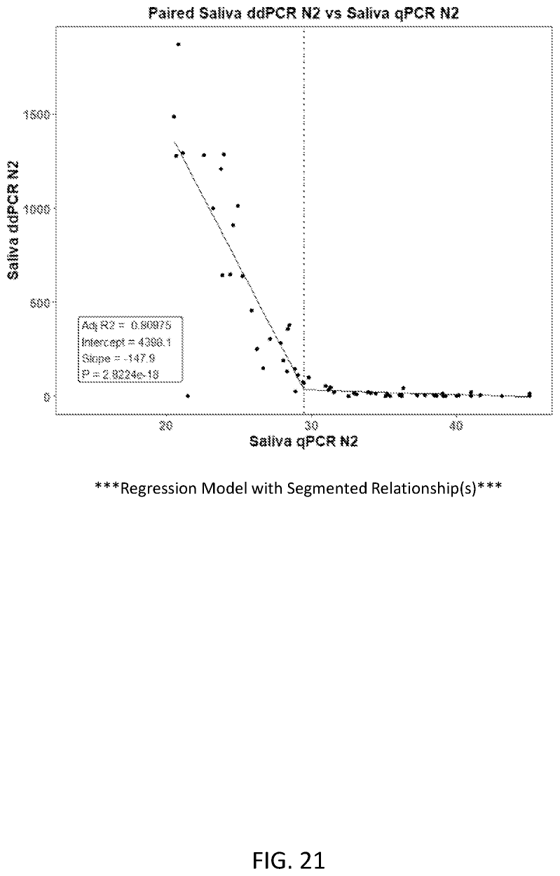

FIG. 21 shows regression modeling comparing paired ddPCR and qPCR of saliva samples using N2-region-targeting primers using the following model:

Y=saliva dd N2, x=saliva q N2

Y=4398.14−147.90*X(X<=29.51)−2.331*X(X>29.51)

Multiple R-Squared: 0.8176, Adjusted R-squared: 0.8097.

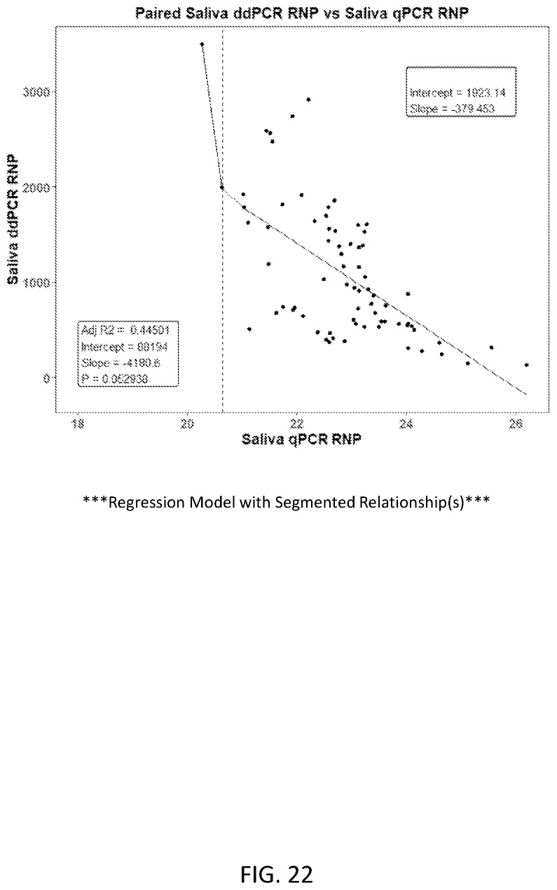

FIG. 22 shows regression modeling comparing paired ddPCR and qPCR of saliva samples using RNP control primers using the following model:

Y=saliva ddPCR RNP, x=saliva_q_RNP

Multiple R-Squared: 0.4194, Adjusted R-squared: 0.3945

If the point with saliva_q_RNP<20 is dropped then

Y=88194−4181*X(X<=20.636)−379.45*X(X>20.636)

Multiple R-Squared: 0.4681, Adjusted R-squared: 0.445.

Importantly, the results show that there is a linear relationship between RT-PCR and ddPCR within a certain range of Ct values (roughly 20-30).

### Example 3—Longitudinal Testing

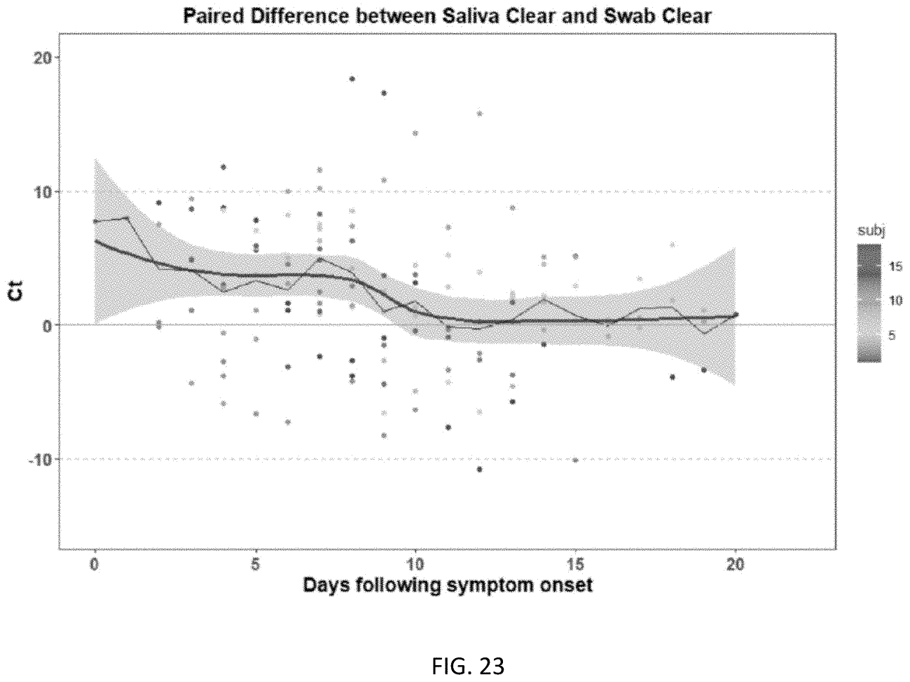

Paired longitudinal saliva and nasal swab samples were obtained from COVID-19 patients at various days after symptom onset. Those samples were tested for viral load using qPCR analysis with Ct values standing as a proxy for viral load. FIG. 23 shows the results of that testing where the y axis represents the difference in Ct values (saliva-nasal swab). While the measured viral load is initially higher in nasal swab samples compared to saliva samples at the time of diagnosis in 19 patients, that difference disappears by day 10. The results show that both nasal swab and saliva are useful sources for monitoring viral load in patients over time.

### INCORPORATION BY REFERENCE

References and citations to other documents, such as patents, patent applications, patent publications, journals, books, papers, web contents, have been made throughout this disclosure. All such documents are hereby incorporated herein by reference in their entirety for all purposes.

### EQUIVALENTS

Various modifications of the invention and many further embodiments thereof, in addition to those shown and described herein, will become apparent to those skilled in the art from the full contents of this document, including references to the scientific and patent literature cited herein. The subject matter herein contains important information, exemplification and guidance that can be adapted to the practice of this invention in its various embodiments and equivalents thereof.

What is claimed is:

1. A method of detecting a viral infection, the method comprising:
providing a saliva sample from a patient;
performing one or more assay(s) to detect viral nucleic acid and/or virus-specific antibodies or antigens in the saliva sample; and
diagnosing the patient as having been infected with the virus upon detection of the viral nucleic acid, or the virus-specific antibodies or antigens in the saliva sample.

2. The method of claim 1 wherein the virus is a respiratory virus.

3. The method of claim 2, wherein the virus is a member selected from the group consisting of paramyxoviridae, picornaviradae, coronaviridae, parvoviridae, and enteroviridae.

4. The method of claim 2, wherein the virus is a virus causative of severe acute respiratory syndrome.

5. The method of claim 4, wherein the virus is a SARS-CoV-2 virus.

6. The method of claim 1, wherein the performing step comprises performing assays to detect viral nucleic acid and virus-specific antibodies in the saliva sample.

7. The method of claim 1, wherein the performing step comprises performing PCR on nucleic acids from the saliva sample using primers specific for viral nucleic acid.

8. The method of claim 7, wherein the viral-nucleic acid-specific primers target one or more of the N, ORF1ab, and E genes.

9. The method of claim 7, further comprising quantifying the viral nucleic acid.

10. The method of claim 9, wherein the quantifying step comprises performing Q-PCR.

11. The method of claim 7, wherein the PCR is digital PCR (dPCR).

12. The method of claim 11, wherein the dPCR is droplet digital PCR (ddPCR).

13. The method of claim 10, further comprising determining severity of the infection based on quantification of viral nucleic acid.

14. The method of claim 7, further comprising comparing viral nucleic acid quantities in a plurality of saliva samples obtained from the patient at successive time points and determining disease progression based on increases or decreases in the viral nucleic quantities over time.

15. The method of claim 7, further comprising assessing a clinical course of treatment based on the viral nucleic acid quantity.

16. The method of claim 15, wherein the clinical course of treatment is selected from the group consisting of intubation, ICU admission, discharge, time until intubation, time until discharge, and death.

17. The method of claim 1, wherein the virus-specific antibodies comprise immunoglobulin A (IgA) or immunoglobulin G (IgG) antibodies.

18. The method of claim 17, wherein the virus-specific antibodies comprise IgA and IgG antibodies.

19. The method of claim 15, further comprising quantifying virus-specific antibodies.

20. The method of claim 19, further comprising assessing a clinical course of treatment based on the quantity of the virus-specific IgA and IgG antibodies.

21. The method of claim 20, wherein the clinical course of treatment is selected from one or more of intubation, ICU admission, discharge, time until intubation, time until discharge, and death.

22. The method of claim 17, further comprising normalizing the quantities of virus-specific IgA and IgG antibodies against total IgA and IgG antibodies in the saliva sample.

23. The method of claim 1, wherein the assay comprises an immunoassay.

24. The method of claim 23, wherein the immunoassay is a biomarker-based assay.

25. The method of claim 24, wherein the biomarker-based immunoassay is an enzyme-linked immunosorbent assay (ELISA), bead-based assay, a luminescent assay, a metal-linked immunosorbent assay, or a point-of-care immunochromatographic assay.
